(19) United States
(12) Patent Application Publication
(10) Pub. No.: US 2004/0068174 A1
(43) Pub. Date: Apr. 8, 2004

(54) Induced body current meter

(76) Inventor: Joseph D. Bowman, Cincinnati, OH (US)

Correspondence Address: KLARQUIST SPARKMAN, LLP, 121 SW SALMON STREET, SUITE 1600, PORTLAND, OR, 97204, US

(21) Appl. No.: 10/468,490

(22) PCT Filed: Apr. 1, 2002
(86) PCT No.: PCT/US02/10527

(51) Int. Cl. A61B 5/055

(52) U.S. Cl. 600/410

(57) ABSTRACT

An induced body current meter uses measurements of extremely low frequency magnetic fields in three spatial axes to determine the maximum current density induced in the brain or other body organ from exposure to the magnetic fields. The method extrapolates from a detailed dosimetry of induced current from magnetic field exposure for a reference body in a reference magnetic field. The meter can be carried or worn to monitor magnetic field exposure of its user. The meter's induced current measurement can be directly compared to induced current health hazard guidelines for health regulation compliance assessment.

### CROSS REFERENCE TO RELATED APPLICATIONS

This application claims the benefit of U.S. Provisional Patent Application No. 60/282,352, filed Apr. 5, 2001.

### TECHNICAL FIELD

This invention relates generally to measurement of extremely low frequency (ELF, =˜3-3000 Hz) magnetic fields in the environment including workplaces, such as for purposes of assessing compliance with magnetic field health hazard standards. The invention more particularly relates to determine induced body currents caused from exposure to such magnetic fields.

### BACKGROUND AND SUMMARY

Based on scientific studies linking exposure to high strength magnetic fields with neurologic and cardiac problems due to induced body currents, an international non-governmental scientific body titled the International Commission for Non-Ionizing Radiation Protection (ICNIRP) has established a magnetic field exposure guideline to protect against these acute health hazards. The ICNIRP guideline recommends against magnetic field exposure that would produce induced body currents of more than 10 mA/m² in the brain and central nervous system.

This ICNIRP induced current guideline has been adopted for purposes of establishing magnetic field health standards by government agencies in many countries, including the European Union, Great Britain, and Germany, among others. In the U.S., the American Conference for Governmental Industrial Hygienists (ACGIH) has adopted the ICNIRP induced current guideline as the basis for establishing a Threshold Limit Value.(TLV) for ELF magnetic fields. The U.S. Occupational Safety and Health Administration (OSHA) uses the TLV to regulate. ELF magnetic field exposures. Accordingly, in these countries, the ICNIRP induced. current guideline determines when the magnetic field environment poses a health risk, and when controls on exposure to such fields are needed. Further, in some of these countries, standards based on the ICNIRP induced current guideline carry the force of law, such that fines or other penalties may be imposed for failure to comply with the standards.

Due to the impracticality of measuring induced current within living persons, ICNIRP and ACGIH have set reference levels for environmental and occupational magnetic fields at which induced body currents may be expected to exceed the ICNIRP induced body current guideline. These reference levels are based on a calculation of the induced current density in a simplified dosimetric model of the human body from a given magnetic field. The ICNIRP calculation is based on using a coil of wire the radius of a person's waist. The ACGIH's calculation uses a homogenous “egg” (oblate spheroid) having a person's height and width. Both agencies use a simplified magnetic field having a single frequency and a linear vertical orientation (or “polarization”). From these simplified models, the agencies derived formulas for the magnetic field reference levels, which depend upon the frequencies present in the magnetic field and the magnetic field magnitudes at those frequencies (i.e., the magnetic field frequency spectrum).

Use of these magnetic field reference levels to assess violation of magnetic field exposure guidelines has several drawbacks. Because the formulas derived for the reference levels depend on the magnetic field magnitude at multiple frequencies, accurate measurement requires an instrument that can measure the magnetic field frequency spectrum. These instruments are costly (e.g., $3,000 and up). Further, accurately measuring the magnetic field frequency spectrum requires extensive training, and a multiple step procedure. The instruments' frequency spectrum measurements are based on digital Fourier transforms, a sophisticated mathematical procedure whose improper use can give erroneous results. In cases where the frequency spectrum measurement indicates only a single frequency is present in the environment, a simpler “gauss meter” costing less than $1,000 can be used to measure the magnetic field magnitude. However, most workplaces have magnetic fields with multiple frequencies.

A further problem is that the ICNIRP and ACGIH magnetic field reference levels may be insufficiently accurate at estimating compliance with the induced body current guideline. Accurate compliance measurements can have very significant impacts in the workplace. Negative errors in measuring guideline compliance can lead to a false decision that there is no health risk. Positive errors can lead to false decision that controls are needed. In either case, compliance measurement inaccuracies can prove costly.

Analysis by the inventor has shown that the ICNIRP and ACGIH magnetic field reference levels may be off by 100% or more at determining compliance with the induced current guideline. The analysis used detailed dosimetry models developed by researchers based on magnetic resonance imaging (MRI) images of an average male body. The MRI-based dosimetry provides induced current data from “standard” magnetic fields, i.e., linearly polarized fields of 60 Hz frequency and 1 μT (microtesla) magnitude aligned along each of the body's three axes (vertical, transverse and lateral). Measurements in a workplace with a “wave capture” instrument (called the Multiwave II® of Electrical Research and Management, State College, Pa.) were used to determine the frequency spectrum, and calculate compliance with the TLV and ICNIRP magnetic field reference levels. For comparison, the induced body current also was estimated using a methodology developed by the inventor from the Multiwave data and the MRI-based dosimetry. In 59 measurements taken in six factories, the ICNIRP reference levels had errors relative to the induced current estimates ranging from −97% to +1%, and the ACGIH's TLV had errors from −10% to +1470%. Although the induced current estimation methodology uses a few mathematical approximations, the induced current estimates are expected to be accurate within ±10%.

The errors in the ICNIRP and ACGIH magnetic field reference levels can result from: (1) multiple magnetic field frequencies, (2) variable spatial orientation of the field, (3) non-linear polarization, (4) variable magnitudes across the body, (5) variable conductivities within the body, and (6) the shape of bodily organs.

The present invention provides a more practical and accurate measurement of compliance with magnetic field health guidelines. An induced body current meter according to the invention provides a reading of the induced body current produced from exposure to an environment's magnetic field. The reading can be compared directly to established body current guidelines. In one implementation of the invention, the induced body current meter includes a multi-axis magnetic field sensor (e.g., a 3-axis gaussmeter), and a programmed processor. The processor performs a calculation estimating the induced body current resulting from exposure to the sensed magnetic field, based on a dosimetry model derived from MRI imaging data of a reference human body. In a representative implementation, the calculation extrapolates the body current induced by the sensed magnetic field in the brain from a multiple-axis numerical dosimetry model of an average adult male body in a 60 Hz magnetic field. Alternative implementations of the meter can be adapted to determine induced body currents in other organs of the body.

In at least some implementations of the invention, the induced body current meter is designed as a mobile instrument that can be worn on the person (e.g., by a work or health inspector) into a monitored environment. For an implementation for measuring induced body current in the head for example, the mobile instrument preferably is carried on the person's head (e.g., with a head band or hard-hat mounting) with gauss-meter induction coils aligned with the dosimetry model axes (e.g., with coils aligned on the vertical, transverse and lateral axes of the body as in the dosimetry model). Alternative implementations for other organs may be carried on the person near the organ in question. Although integrated in single portable unit housing in some implementations, the magnetic field sensor and programmed processor portions of the instrument can be housed separately in other implementations (e.g., separate portable magnetic field sensor unit and programmed computer for the induced body current calculation).

Implementations of the meter can provide a read-out of the induced body current measurement on a display, or log measurement data to a recording media (e.g., in an electronic memory, magnetic recording medium, printed medium, or other recording media) for later analysis. Some implementations can provide a peak hold feature for obtaining an upper bound of the exposure measurement.

Additional features and advantages will be made apparent from the following detailed description of the illustrated embodiment which proceeds with reference to the accompanying drawings.

### BRIEF DESCRIPTION OF THE DRAWINGS

FIG. 1 is a flow diagram of an induced body current meter instrument in accordance with one implementation of the invention.

### DETAILED DESCRIPTION

In the following detailed description, one implementation of measuring induced body current exposure from magnetic fields according to the invention is embodied in an induced body current meter 100. The meter 100 utilizes a multiple axis sensing of the magnetic field of an environment, and extrapolates the induced body current from exposure to that field based upon a detailed, empirically-derived dosimetric model of the human body. This yields an induced body current value for ready comparison against established induced body current guidelines for health hazard compliance assessment. The induced body current determination also is expected to have higher accuracy comparative to prior magnetic field health hazard assessment methodologies.

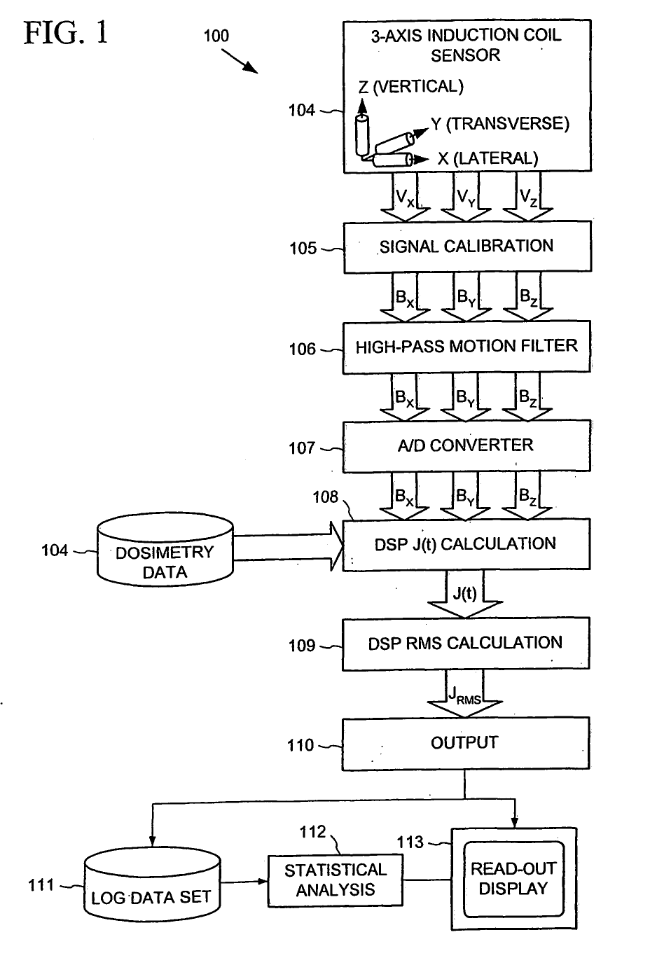

With reference to FIG. 1, a suitable hardware platform for the meter 100 includes a multiple-axis magnetic field sensor 104, signal processing circuitry 105-109 for calculating the induced body current from the sensed magnetic field signals, and an induced body current measurement output 110. An example of such platform is a programmable three-axis gaussmeter, such as the currently available EMDEX PAL™ from Enertech Consultants of Campbell, Calif., USA. The programmable three-axis gaussmeter is modified via programming to perform processing of the sensed magnetic field signals to yield the induced body current measurement. In alternative implementations of the meter 100, a purpose-built hardware platform can be used. Further, although the exemplary meter 100 is housed in a single mobile unit, alternative implementations of the meter 100 can have any variety of configurations, such as a handheld, laptop or other mobile computer in communication with a magnetic field sensor peripheral component; or a mobile magnetic field sensor unit for recording or transmitting magnetic field data and operated in combination with a stationary desktop, workstation or server computer that performs the induced body current measurement calculations to provide the induced body current measurements according to the invention.

A suitable multiple-axis magnetic field sensor 104 for the meter 100 is a three-axis induction coil sensor, which has three induction coils with ferrite cores in an orthogonal axis configuration (with axes denoted x, y and z corresponding to lateral (left-to-right), transverse (front-to-back) and vertical axes of the body, respectively). The induction coils each produce a voltage signal (i.e., Vₓ(t), V_{y}(t), and V_{z}(t)) related to the magnetic field's derivative in the direction of its respective axis. Alternative implementations of the meter 100 can employ different magnetic field sensor technologies that produce signals indicative of the magnetic field sensed in multiple spatial dimensions, whether in 3 orthogonal axes or other configuration.

In the signal processing circuitry 105-109, signal calibration and conditioning circuitry 105 first calibrates the sensor voltage signals to ensure proportionality with magnetic field measurement units, i.e., calculates the magnetic field value for each axis as a calibration function (F) of the sensor voltage signals as shown in the following relation:

dBᵢ(t)/dt=F[Vᵢ(t)], for i=x, y, z.

The circuitry 105 can include dynamic ranging. In alternative implementations of the meter 100, the digital signal processor 108 can be programmed to perform some or all of the calibration function (F).

The meter 100 next includes a motion filter 106, which filters out the contribution to the magnetic field signal (dBᵢ(t)/dt) due to the meter's motion through gradients in the earth's magnetic field, while passing contributions from AC electrical equipment and other higher frequency magnetic field sources in the environment. A suitable motion filter 106 is a high-pass filter set to pass 40 Hz and higher signals. Although the biological effects of induced body currents have not been shown to have any frequency dependency, gaussmeters designed for cancer research commonly include such motion filters as a standard feature. The meter 100 includes the motion filter 106 so that the magnetic field sensor signal is comparable to a standard gaussmeter signal. Some implementations of the meter 100 include a switch to selectively bypass the motion filter 106 (i.e., remove the motion filter from the signal path) in case it is desired to also measure induced body currents resulting from motion.

Following the motion filter 106, the meter 100 has an analog-to-digital converter 107 for converting the analog magnetic field sensor signal into digital data for processing by a digital signal processor 108-109. The sampling rate of the converter 107 should be at least twice the upper bound of the instrument's flat frequency response. Since some existing gaussmeters have an upper bound of 800 Hz for example, this implies a sampling rate of around 1600 Hz. However, because violations of the induced current guideline can occur with induction heaters that operate at much higher frequencies, the meter 100 preferably provides a broader frequency range and higher sampling rate for measuring induced current guideline compliance. The output of the analog-to-digital converter 107 are the three components of the magnetic field derivative vector dB_{α}(t)/dt (where α=x, y, z or lateral, transverse and vertical) as a function of time (t).

The digital signal processor (DSP) in the meter 100 processes the magnetic field derivative vector data in an induced current density calculation 108 to produce a measurement of the body current induced in the brain from exposure to the sensed magnetic field. The digital signal processor is programmed with data 104 from an MRI-based dosimetry model for use as parameters of the induced body current density calculation. The dosimetry parameters are obtained from magnetic resonance imaging (MRI) of an adult male body. The parameters are the x, y and z vector components of current density ^{α}J calculated for a 1 μT peak, 60 Hz magnetic field applied along the respective α axis (where α=x, y, z).

Exemplary MRI-based dosimetry parameters. for induced brain currents are listed in the following Table 1. Because the ICNIRP induced current guideline is based on neurological disturbances, induced brain current measurement using the listed parameters is currently preferred for health hazard compliance assessments. However, the meter 100 can be reprogrammed with dosimetry parameters for induced current in other body organs, such as in the case that future research identifies health risks from induced currents in such other organs (e.g., heart, uterus, whole body, etc.).

TABLE 1

| | ^{α}J_{β} [μA/m²] | | |
|---|---|---|---|
| Magnetic field axis | β = x | β = y | β = z |
| x | −0.0145 | 0.3611 | 0.1028 |
| y | −0.3315 | 0.0018 | −0.0100 |
| z | −0.0557 | 0.0289 | 0.0003 |

In one implementation, the DSP is programmed to perform the calculation 108 in accordance with the following Equation 1 of the induced current density J(t) as a function of time using the dosimetry parameters 104 and magnetic field derivative vector data from the converter 107.

$$J ( t ) = \frac{1}{2 π \times 1 µT \times 60 Hz} \sqrt{{\sum }_{α = x , y , z} {( {\sum }_{β = x , y , z} {}_{}^{α}{J}{}_{β}^{} \frac{ⅆ {B}_{α} ( t )}{ⅆ t} )}^{2}} \qquad ( 1 )$$

The meter's digital signal processor further performs a calculation 109 of the root-mean-square (RMS) value (J_{RMS}) of the just calculated induced current density. The averaging time of the RMS calculation 109 should depend on the biologic response time. A suitable averaging time for the meter 100 is several cycles of 50/60 Hz AC electricity, or about 0.1 seconds.

The just described calculations 108-109 in this implementation of the meter 100 have the advantage of not involving measurement of the frequency spectrum, and thus does not require calculation of the Fourier or other complex mathematical transform. This can reduce the cost of the meter (e.g., because higher cost/speed components needed to process such transforms are not needed), and can reduce the likelihood of error from improper use.

The meter 100 finally provides an output 110 of the induced body current measurement. In one implementation, the meter 100 provides a variety of output options, including via a data logger, a direct digital read-out, and read-out with peak hold. With the data logger option, the meter 100 stores a data set in an on-board digital memory 111 containing the J_{RMS} measurement taken at periodic intervals (e.g., every 1-10 seconds), along with the time of measurement. Via user selection, the meter 100 can be set to display various statistics (produced from a statistical analysis 112) from the logged measurements on a meter display 113, such as average and maximum. Also, the data set can be downloaded from the meter 100 to a computer (e.g., laptop, desktop, handheld, server, etc.) for further numerical analysis and graphing (e.g., using a spreadsheet or like software application).

The meter 100, with the direct digital read-out option, displays the J_{RMS} measurement on the meter's display 113 (e.g., on a digital LCD display screen). Alternatively, with the peak hold read-out option, the meter 100 captures and displays a peak (maximum) value of the induced current measurement since last reset or other time interval. The peak hold option is particularly appropriate for obtaining the upper bound of the magnetic field-induced current exposure within an environment for health hazard compliance decisions.

For use in induced current guideline compliance assessment, the meter 100 can be carried or worn on the person, e.g., of a worker in course of work duties or an inspector on a walk-through of a work place or other strong magnetic field environment. Preferably, the meter is carried near the head, such as attached on a head band or mounted on a hard hat. The meter 100 is oriented with the induction coils of the sensor 104 aligned to the vertical, transverse and lateral axes of the person's body, as in the dosimetry model. In this way, the meter induced body current measurement more closely reflects the person's actual exposure in the environment. In implementations of the meter adapted for measuring induced current exposure of other body organs (e.g., programmed with dosimetry parameters for the heart, uterus, or other organs), the meter can be carried or worn near such other organ of the person, again with the sensor appropriately aligned with the dosimetry model axes.

Having described and illustrated the principles of our invention with reference to an illustrated embodiment, it will be recognized that the illustrated embodiment can be modified in arrangement and detail without departing from such principles. It should be understood that the programs, processes, or methods described herein are not related or limited to any particular type of computer, sensor and signal processing hardware platform. Various types of general purpose or specialized hardware may be used with or perform operations in accordance with the teachings described herein. Elements of the illustrated embodiment shown in software may be implemented in hardware and vice versa.

In view of the many possible embodiments to which the principles of our invention may be applied, it should be recognized that the detailed embodiments are illustrative only and should not be taken as limiting the scope of our invention. Rather, we claim as our invention all such embodiments as may come within the scope and spirit of the following claims and equivalents thereto.

We claim:

1. A device for determining induced current exposure of a body organ in a magnetic field, comprising:
a three-axis magnetic field sensor for measuring an environmental magnetic field sensed on three body axes and thereby producing magnetic field signals;
a signal processor for processing the magnetic field signals in accordance with an theoretically-derived, empirically-based dosimetry model of the body organ in a reference body exposed to a reference magnetic field on the three body axes to determine a value of the induced current in the body organ from exposure to the environmental magnetic field; and
an induced body current output for providing a representation of the induced current value for further analysis or viewing.

2. The device of claim 1 wherein the signal processor calculates the induced current density value in the body organ based on the measured magnetic field and dosimetry data derived from magnetic resonance imaging of the reference body in the reference magnetic field.

3. The device of claim 2 wherein the dosimetry model uses the reference magnetic field resulting from alternating current (AC) electricity with a 60 Hz frequency.

4. The device of claim 2 wherein the dosimetry model is valid for magnetic fields with frequencies up to 100 kHz, including the extremely low frequency band (3-3000Hz) and the very low frequency band (3-30 kHz).

5. The device of claim 2 wherein the calculation includes calculating an induced current density J(t) from sensed magnetic field derivative vector components dB_{α}(t)/dt and current density dosimetry parameters ^{α}J_{β} for each of three body axes α,β=x, y and z.

6. The device of claim 5 wherein the calculation further includes calculating a root-mean-square value J_{RMS} of the induced current density J(t).

7. The device of claim 5 wherein the calculating the induced current density J(t) is performed according to the following relation:
$$J ( t ) = \frac{1}{2 π \times 1 μ T \times 60 Hz} \sqrt{{\sum }_{α = x , y , z} {( {\sum }_{β = x , y , z} {}_{}^{α}{J}{}_{β}^{} \frac{ⅆ {B}_{α} ( t )}{ⅆ t} )}^{2}} .$$

8. The device of claim 7 wherein the current density dosimetry parameters ^{α}J_{β} for the body organ being the brain are substantially those given in the table:

| | ^{a}J_{β}[μA/m²] | | |
|---|---|---|---|
| Magnetic field axis | β = x | β = y | β = z |
| x | −0.0145 | 0.3611 | 0.1028 |
| y | −0.3315 | 0.0018 | −0.0100 |
| z | −0.0557 | 0.0289 | 0.0003 |

9. The device of claim 7 wherein the current density dosimetry parameters ^{α}J_{β} for the body organ being the brain are within a range of about ±10% of those given in the table:

| | ^{a}J_{β}[μA/m²] | | |
|---|---|---|---|
| Magnetic field axis | β = x | β = y | β = z |
| x | −0.0145 | 0.3611 | 0.1028 |
| y | −0.3315 | 0.0018 | −0.0100 |
| z | −0.0557 | 0.0289 | 0.0003 |

10. The device of claim 7 wherein the current density dosimetry parameters ^{α}J_{β} are for the body organ being one of the following: the brain, the heart, the uterus, and the whole body.

11. The device of claim 5 wherein the x, y and z body axes are lateral, transverse and vertical axes of the reference body.

12. The device of claim 1 wherein the three body axes are lateral, transverse and vertical axes of the reference body.

13. The device of claim 1 wherein the output provides a digital data log of the induced current value.

14. The device of claim 1 wherein the output provides a peak value of the induced current value in a time period.

15. A method of use of the device of claim 1 comprising carrying the three-axis magnetic field sensor on the person of a user in proximity to the body organ of the user and in alignment with the body axes of the user.

16. The method of claim 15 wherein the three body axes are lateral, transverse and vertical axes of the reference body and of the user.

17. A method of determining induced current exposure of a body organ in a magnetic field, comprising:
producing signals relating to magnetic field derivative vectors dB_{α}(t)/dt on three axes α=x, y, z from sensing a magnetic field aligned on each of the axes in an environment;
calculating an induced current density J(t) as a function of the magnetic field derivative vectors dB_{α}(t)/dt and a set of empirically-based dosimetric current density parameters ^{α}J_{β} representing the contribution to the induced current density in the body organ on each of the axes a from a reference magnetic field on each of the vector components β; and
providing an output measurement based on the calculated induced current density J(t).

18. The method of claim 17 further comprising:
calculating a root-mean-square value of the induced current density J(t); and
wherein the output measurement includes the root-mean-square value.

19. The method of claim 17 wherein the calculating the induced current density J(t) is performed according to the following relation:
$$J ( t ) = \frac{1}{2 π \times 1 μ T \times 60 Hz} \sqrt{{\sum }_{α = x , y , z} {( {\sum }_{β = x , y , z} {}_{}^{α}{J}{}_{β}^{} \frac{ⅆ {B}_{α} ( t )}{ⅆ t} )}^{2}} .$$

20. The method of claim 19 wherein the current density dosimetry parameters ^{α}J_{β} are substantially those given in the table:

| | ^{a}J_{β}[μA/m²] | | |
|---|---|---|---|
| Magnetic field axis | β = x | β = y | β = z |
| x | −0.0145 | 0.3611 | 0.1028 |
| y | −0.3315 | 0.0018 | −0.0100 |
| z | −0.0557 | 0.0289 | 0.0003 |

21. The method of claim 19 wherein the current density dosimetry parameters ^{α}J_{β} are within a range of about ±10% of those given in the table:

| | ^{a}J_{β}[μA/m²] | | |
|---|---|---|---|
| Magnetic field axis | β = x | β = y | β = z |
| x | −0.0145 | 0.3611 | 0.1028 |
| y | −0.3315 | 0.0018 | −0.0100 |
| z | −0.0557 | 0.0289 | 0.0003 |

22. The method of claim 17 wherein the current density dosimetry parameters ^{α}J_{β} are for one of the following body organs: the brain, the heart, and the uterus.

23. An induced body current meter, comprising:
a three-axis induction coil magnetic field sensor operative to produce measured magnetic field signals relating to a characteristic of the magnetic field of an environment on each of three spatial axes;
a signal calibrator operative to calibrate the sensed magnetic field signals; and
a signal processor operative to produce a determination of an induced current in a body organ based on the sensed magnetic field signals and a dosimetry model of the body organ in a reference body exposed to a reference magnetic field on the three spatial axes.

24. The induced body current meter of claim 23 wherein the signal processor performs a calculation to extrapolate the determination of the induced current density in the body organ based on dosimetry data derived from magnetic resonance imaging of the reference body in the reference magnetic field.

25. The induced body current meter of claim 23 further comprising:
a motion filter operative to filter a frequency component portion of the sensed magnetic field signals contributed by motion of the meter through gradients of the earth's magnetic field, and pass other component portions contributed by extremely low frequency environmental magnetic fields; and
a switch for selectively applying or bypassing the motion filter from a sensed magnetic field signal path of the induced body current meter.

26. A three-axis programmable gaussmeter, comprising:
a three-axis magnetic field sensor for producing sensor signals relating to a characteristic of the magnetic field of an environment on each of three spatial axes;
a digital-to-analog converter for digitizing the sensor signals; and
a digital signal processor programmed to process the sensor signals in accordance with an equation relating a measured environmental magnetic field on the three spatial axes together with dosimetric data modeling contributions to an induced current density in a reference body organ from exposure to a reference magnetic field on the three spatial axes to determine the induced current density in the body organ from the environmental magnetic field; and
an induced body current output.

27. The three-axis programmable gaussmeter of claim 26 wherein the digital signal processor is further programmed to calculate a root-mean-square value of the determined induced current density for output by the induced body current output.

28. The three-axis programmable gaussmeter of claim 26 wherein the induced body current output provides a peak hold indication representing a peak of the determined induced current density in the body organ over a time period.

29. The three-axis programmable gaussmeter of claim 26 wherein the digital signal processor is programmed to calculate an induced current density J(t) as a function of the magnetic field derivative vector components dB_{α}(t)/dt and a set of empirically-based dosimetric current density parameters ^{α}J_{β} representing the contribution to the induced current density in the body organ on each of the body axes α from a reference magnetic field on each of the spatial axes β in accordance with the following relation:
$$J ( t ) = \frac{1}{2 π \times 1 μ T \times 60 Hz} \sqrt{{\sum }_{α = x , y , z} {( {\sum }_{β = x , y , z} {}_{}^{α}{J}{}_{β}^{} \frac{ⅆ {B}_{α} ( t )}{ⅆ t} )}^{2}} .$$
